(19) United States
(12) Patent Application Publication
(10) Pub. No.: US 2017/0020707 A1
(43) Pub. Date: Jan. 26, 2017

(54) Elastic Strip for Holding the Ankle of a Person and Ankle Orthosis

(71) Applicant: GIBAUD, Saint Etienne (FR)

(72) Inventors: Guillaume DUPORT, Saint Etienne (FR); Frederic BAUDOUIN, Saint Priest en Jarez (FR)

(21) Appl. No.: 15/216,898

(22) Filed: Jul. 22, 2016

(30) Foreign Application Priority Data
Jul. 23, 2015 (FR) 15/57005

(51) Int. Cl. A61F 5/01

(52) U.S. Cl. CPC A61F 5/0111

(57) ABSTRACT

An elastic strip for holding the ankle of a person as well as an ankle orthosis comprising an ankle brace includes a first portion extending from a first end of the strip and intended to be wound around the foot; a second portion extending in the extension of the first portion to a second end of the strip, intended to be wound around the ankle; a first attachment component, located on the outer face of the strip, at the junction between the first and second portions;
and a second attachment component intended to cooperate with the first attachment component, located on the inner face of the strip, at the second end of the strip. The attachment components are disc-shaped and the second attachment component has a diameter lower than or equal to the diameter of the first attachment component.

### FIELD OF THE INVENTION

The present invention relates to an elastic strip for holding the ankle of a person as well as an ankle orthosis comprising an ankle brace and such a strip.

### BACKGROUND

It is known to equip a person with a sprain of the ankle of an orthosis that comprises:
on the one hand, an ankle brace including a tubular element made of elastic material intended to be threaded over the ankle in the manner of a sock, the ankle brace having a compression function intended to reduce a possible edema;
and on the other hand an elastic holding strip, also called “strapping strip”, which is intended to be wound over the ankle brace and whose function is to prevent the setting in varus of the ankle.

An ankle orthosis of the prior art is illustrated in FIGS. 1 to 3.

This orthosis 100 comprises an ankle brace 101 shown threaded on the ankle of a person, and an elastic holding strip 102 wound over the ankle brace 101. The strip 102 comprises at one first end an assembly component 103 which cooperates with a complementary assembly component (not visible) located on a front area of the ankle brace 101, on the top of the foot. The strip 102 further comprises a first attachment component 104, located on the outer face of the strip, in the median portion of the strip 102, and a second attachment component 105, located on the inner face of the strip, in the vicinity of the second end of the strip 102. In this known orthosis, the assembly component 103 as well as the first and second attachment components 104, 105 have a rectangle shape.

The setting up of the strip 102 on the ankle brace 101 is performed as follows.

Initially, the strip 102 is assembled by its first end to the ankle brace 101, thanks to the assembly component 103 (FIG. 1). Then one starts winding the strip 102 by stretching it, at first, around the foot of the user. The first attachment component 104 is then located on the top of the foot (FIG. 2). The winding of the strip 102 is continued, around the ankle and the heel. Finally, the strip 102 is fastened on itself, by cooperation between the first and second attachment components 104, 105 (FIG. 3).

Depending on how the strip is wound and refolded on itself, the second attachment component 105 may not be exactly superimposed on the first attachment component 104: thus, as illustrated in FIG. 3, the two attachment components 104, 105 may form an angle therebetween. This is not satisfactory.

Indeed, on the one hand, an imperfect superposition of the two attachment components may result in an insufficient attachment force, hence a risk that the strip 102 comes off, in particular when the user moves the foot.

But, on the other hand, the user instinctively seeks to match the two attachment components. But if the strip was not properly wound around the foot and/or the ankle, it is difficult to superimpose the two attachment components. In order to achieve this, the user must asymmetrically pull on the strip in order to bring the second attachment component to be placed exactly on the first attachment component. It follows that a longitudinal area of the strip is excessively stretched, which may cause discomfort for the user, while another longitudinal area of the strip is not stretched enough, and may form folds which are also a discomfort source. Furthermore, such a positioning may be insufficiently efficient in terms of the compression.

### SUMMARY

The present invention aims at overcoming the drawbacks mentioned hereinabove.

To this end, and according to a first aspect, the invention relates to an elastic strip for holding the ankle of a person, the strip having an inner face and an outer face and comprising:
a first portion which extends from a first end of the strip and which is intended to be wound substantially around the foot;
a second portion which extends in the extension of the first portion to a second end of the strip and which is intended to be wound substantially around the ankle;
a first attachment component, located on the outer face of the strip, substantially at the junction between the first portion and the second portion;
a second attachment component intended to cooperate with the first attachment component, located on the inner face of the strip in the vicinity of the second end of the strip.

According to a general definition of the invention, the first and the second attachment components are substantially disc-shaped. Furthermore, the second attachment component has a diameter D22 lower than or equal to the diameter D21 of the first attachment component.

Due to the use of this particular shape for the two attachment components, namely the disc, it is possible to centrally superimpose the first and second attachment components with respect to each other, even with different winding configurations of the strip. More precisely, regardless of the angle that the second portion of the strip makes with respect to the first portion at the end of the winding operation, the first and second attachment components, being disc-shaped and having then an infinity of axes of symmetry unlike a rectangle for example, may be placed one upon the other in a substantially perfectly coaxial manner.

Furthermore, the fact of providing first and second disc-shaped attachment components allows providing a reference to the user for a proper positioning of the strip. Indeed, the user must place the second attachment component on the first attachment component, in a substantially coaxial manner. The centering is facilitated by the fact that the second attachment component has the same diameter as the first attachment component or a smaller diameter, the first attachment component thus always being partially visible when the second attachment component is superimposed thereto. Insofar as it is not provided an attachment area of large dimensions on which the second attachment component may be attached in different locations, the invention allows forcing the user to come place the second attachment component on the first attachment component, that is to say in a precise place allowing obtaining the appropriate holding and degree of tightening.

The strip according to the invention thus allows an adaptation to all angulations and morphologies as well as a use on the right foot or the left foot. By guiding the user and allowing an excellent positioning regardless of the angle of arrival of the second portion of the strip, the invention allows setting up the strip without excessive force on the strip or on the attachment component. The comfort of use of the strip is increased because it is more intuitive, and easier. The comfort of use is also improved as the system avoids the folds of the strip or traction surges which may cause discomfort.

Concretely, D22 may be substantially equal to D21, or D22 strictly lower than D21. However, it is understood that the second attachment component must have a diameter large enough to provide a sufficient attachment. In practice, it may be envisaged that D22 is greater than 0.3×D21.

Advantageously, it is envisaged that D22>0.6×D21, preferably D22>0.7×D21, and preferably D22>0.8×D21.

According to one possible embodiment, the first and second attachment components may have substantially identical diameters. “Substantially identical” means with a margin of about 10 or even 5%, that is to say that 0.9×D21<D22<D21, or even that 0.95×D21<D22<D21.

According to one possible embodiment, the strip is substantially rectangular, the diameter of the first and second attachment components being substantially equal to the width of the strip. Herein again, the term “substantially equal” means with a margin of about 10 or even 5%. Providing a first and a second disc-shaped attachment components having substantially the same diameter allows providing to the user with a very precise reference for proper positioning of the strip.

According to another embodiment, the width of the second portion shifts from a value I in the vicinity of the first portion to a value I′ lower than I in the vicinity of the second end. Furthermore, the diameter D21 of the first attachment component is substantially equal to I, and the diameter D22 of the second attachment component is substantially equal to I′. Herein again, the expression “substantially equal” means with a margin of about 10 or even 5%.

The first and second attachment components may be of the hook and loop type of a Velcro® system. For example, the first attachment component may consist of Velcro® loops.

The strip may further comprise, in the vicinity of its first end, and on its inner face, an assembly component on an ankle brace including a tubular element made of elastic material. Unlike attachment components, this assembly component may have a rectangular shape, this ensuring a correct positioning of the starting point of the strip in order to enable an appropriate subsequent winding.

It may be envisaged that the second portion of the strip has a longitudinal opening intended to accommodate the heel.

According to one possible embodiment, the second portion of the strip is substantially flat in the unstressed condition. With this disposition, when the strip is wound around the ankle of the user and held in this position by any suitable means, the heel being housed in the opening, the second portion of the strip matches the shape of the foot without forming a protruding nose. When set up on a person, the strip is thus not troublesome and remains very discreet.

By “unstressed condition”, it is meant the configuration of the strip when it is placed on a support such as a table, without being wound, refolded on itself or stretched. Conversely, the “position of use” means the configuration of the strip when it is placed around the ankle of a person.

According to one possible embodiment, the strip is formed of two longitudinal straps disposed side by side so that a longitudinal edge of a strap is adjacent to a longitudinal edge of the other strap at least on the first portion of the strip, said longitudinal edges being assembled to each other substantially over the entire first portion of the strip, the straps being assembled to each other in the vicinity of the free end of the second portion, and being free with respect to each other—that is to say not assembled—on substantially all of the remainder of the second portion to form a slot therebetween. Said longitudinal edges are for example assembled by sewing without overlapping. In other words, the straps are assembled edge to edge, at least on the first portion of the strip.

According to a first variant, the straps are also assembled edge to edge in the vicinity of the free end of the second portion, and the strip has a substantially constant width.

According to a second variant, the straps are assembled with overlapping in the vicinity of the free end of the second portion, and the strip has a width which decreases towards its second end.

According to a second aspect, the invention relates to an ankle orthosis which comprises an ankle brace including a tubular element made of elastic material intended to be threaded over the ankle of a person, as well as a strip as described hereinabove, the strip being intended to be wound over the ankle brace.

### BRIEF DESCRIPTION OF THE DRAWINGS

Now, it will be described, by way of limiting examples, possible embodiments of the invention, with reference to the appended figures:

FIGS. 1 to 3 show an orthosis of the prior art;

FIG. 4 is a view of a strip according to a first embodiment of the invention, in the unstressed condition;

FIG. 5 is a perspective view of an ankle brace intended for an orthosis according to the invention;

FIG. 6 is a view of a strip according to a second embodiment of the invention, in the unstressed condition;

FIGS. 7 and 8 show two successive steps of setting up the orthosis according to the invention;

FIGS. 9 and 10 show the strip in position of use, in two different configurations.

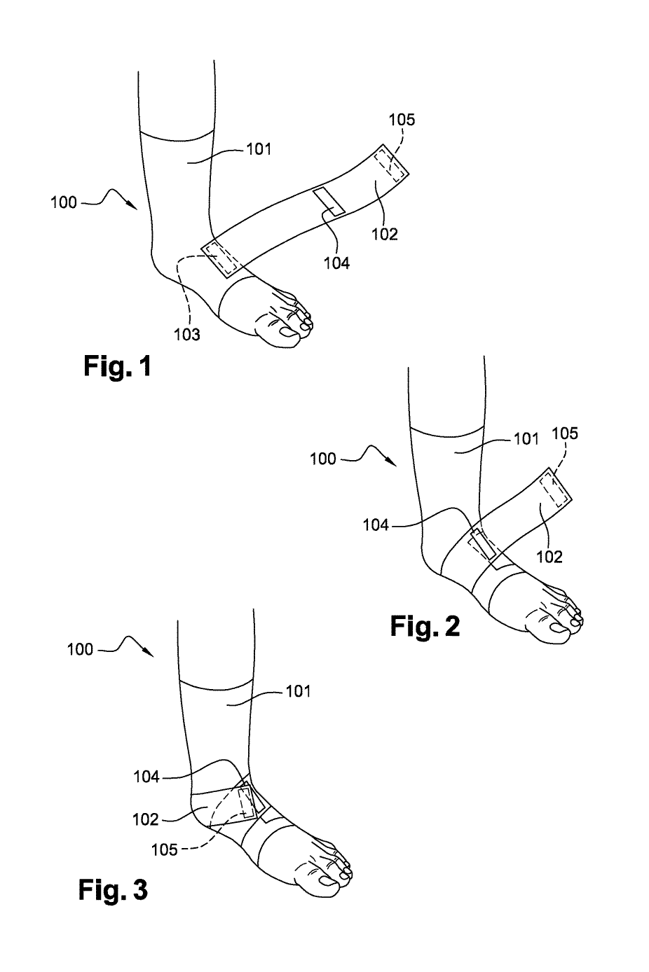

### DETAILED DESCRIPTION OF VARIOUS EMBODIMENTS

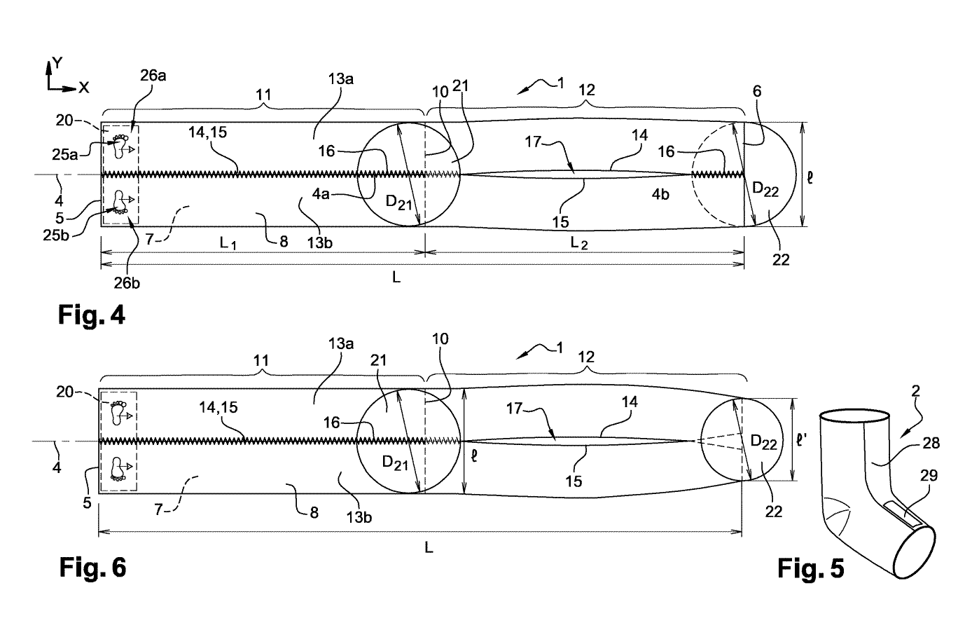

FIG. 4 shows an elastic strip 1 for holding the ankle of a person. This strip 1 is stretchable, at least along a longitudinal direction X, and may be woven, knitted, made from a laminated complex, etc. It is intended to be set up on an ankle brace 2, such as that illustrated in FIG. 5, in order to form an ankle orthosis 3, which is shown in the position of use, on a person, in FIGS. 9 and 10.

In the first embodiment shown in FIG. 4, the strip 1 has substantially the shape of a rectangle of length L which may be in the range of 30 to 50 cm depending on the sizes, and the width 1 which may be in the range of 5 to 10 cm. The longitudinal direction X is defined as the direction of the length of the rectangle, and the transverse direction Y as the direction of the width of the rectangle. The strip 1 has a longitudinal axis of symmetry 4, as well as a first end 5 forming a transverse edge, and a second end 6 forming the opposite transverse edge.

The strip 1 has an inner face 7, which is the one that will face the foot of the user in the position of use, and an opposite outer face 8. In FIG. 4, the strip 1 is shown in the unstressed condition, its outer face 8 being its visible face.

The strip 1 includes:
a first portion 11 which is intended to be wound substantially around the foot of the user. The first portion 11 extends over the entire width of the strip 1, from the first end 5 and over a length L1;
and a second portion 12 which is intended to be wound substantially around the ankle of the user and to envelop the heel. The second portion 12 is adjacent to the first portion 11 and located in the extension thereof, along direction X. The second portion 12 extends over the entire width of the strip 1, to the second end 6 of the strip 1, over a length L2.

The first end 5 of the strip 1 thus forms the free end of the first portion 11, and the second end 6 of the strip 1 forms the free end of the second portion 12. It is further defined the junction line 10 as the transverse edge common to the first and second portions 11, 12. The junction line 10 may be fictive, that is to say does not consist of a concrete assembly line between two initially separate parts.

The first portion 11 of the strip 1 may for example extend over a length L1 of about 50 to 70% of the total length L of the strip 1, for example over a length of about 60% of the total length L of the strip 1. The values of L1 and L2 with respect to L must be determined depending on the anatomy of a person, and of the size of the orthosis 3 which generally belongs to a range including different sizes.

The strip 1 further comprises attachment means of the strip 1 on itself in the position of use. These means comprise:
a first attachment component 21, located on the outer face 8 of the strip 1, substantially at the junction between the first portion 11 and the second portion 12. In other words, the first attachment component 21 is located substantially at the junction line 10, that is to say at the end of the first portion 11 which is contiguous with the second portion 12;
a second attachment component 22 intended to cooperate with the first attachment component 21, located on the inner face 7 of the strip 1, in the vicinity of the second end 6 of the strip 1. In the shown embodiment, the second attachment component 22 projects beyond the second end 6, this embodiment not being limiting.

Typically, the first and second attachment components 21, 22 may be one the loops and the other the hooks of a Velcro®-type system. It may be envisaged that the loops are located on the first attachment component 21.

According to the invention, the first and second attachment components 21, 22 are both substantially disc-shaped. Furthermore, in this first embodiment, the first and second attachment components 21, 22 substantially have the same diameter, D21 and D22 respectively. This diameter may be substantially equal to the width 1 of the strip 1. This embodiment is however not limiting. Thus, it could be envisaged that D22 is lower than D21, for example greater than 0.6×D21, and preferably greater than 0.7×D21, or even greater than 0.8×D21.

In the embodiment shown by way of example, the strip 1 is formed of two substantially identical longitudinal straps 13a, 13b, of length L and width ½. The two straps 13a, 13b are disposed side by side so that a longitudinal edge 14 of a strap 13a is adjacent to a longitudinal edge 15 of the other strap 13b. Insofar as the two straps 13a, 13b are herein identical, said longitudinal edges 14, 15 extend substantially along the axis 4.

The longitudinal edges 14, 15 are assembled to each other over substantially the entire first portion 11 of the strip 1, and in the vicinity of the second end 6 of the strip 1. This assembly may preferably be made edge to edge, that is to say, without overlapping, by a sewing 16.

In contrast, the longitudinal edges 14, 15 are free with respect to each other—that is to say not assembled—on at least the median area of the second portion 12. Thus, a longitudinal opening 17 is formed between these longitudinal edges 14, 15. Insofar as the edges 14, 15 are adjacent, this opening 17 is in the form of a slot which may be very narrow when the strip 1 is in the unstressed condition, but which may move away when the strip 1 is set up on the user, so as to accommodate the heel.

In the embodiment shown by way of example, the second portion 12 of the strip 1 is substantially flat in the unstressed condition. Advantageously, the first portion 11 is also substantially flat in the unstressed condition, the strip 1 being thus flat and in its entirety, as illustrated in FIG. 4.

Furthermore, the strip 1 comprises, near its first end 5, and on its inner face 7, an assembly component 20 of the ankle brace 2.

As illustrated in FIG. 5, the ankle brace 2 includes a tubular element 28 made of elastic material (such as woven, knitting, laminated composite, etc.). The element 28 is intended to be threaded on the ankle of a person, in the manner of a sock. This element 28 is preferably shaped in an ergonomic way, that is to say by forming an angle which will follow the angle formed by the ankle in the position of use. Furthermore, this element 28 is preferably open, of course at its upper end, but also at its lower end.

The ankle brace 2 may preferably include an assembly component 29 complementary to the assembly component 20 provided on the strip 1. This complementary assembly component 29 is arranged on a front area of the ankle brace 2 intended to be placed on the top of the user foot. In the embodiment shown, the assembly component 29 is not located on the plane of symmetry of the ankle brace 2 but slightly offset relative thereto.

It may be for example envisaged that the assembly component 20 is of the hook type and that the complementary assembly component 29 is of the Velcro® system loops type. The system 1 may thus be assembled on the ankle brace 2 and wound around the ankle of the user, above the ankle brace 2.

FIG. 6 illustrates a second embodiment of the strip 1. The latter differs from the first embodiment in the following points.

On the one hand, the second attachment component 22 has a diameter D22 strictly lower than the diameter D21 of the first attachment component 21. For example, it may be envisaged that 0.6 D21<D22<0.9 D21, preferably 0.7 D21<D22 and/or D22<0.8 D21.

On the other hand, in the second portion 12 of the strip 1, the two longitudinal straps 13a, 13b are superimposed in the vicinity of the second end 6 of the strip 1, so that the width of the strip 1, in this second portion, gradually decreases, shifting from I at the junction line 10 to I′<1 at the second end 6. It may be, in particular, envisaged that I′ is substantially identical to D22.

By way of example, I may be close to 6.5 cm, D21 close to I, and F=D22 comprised between 4 and 5 cm.

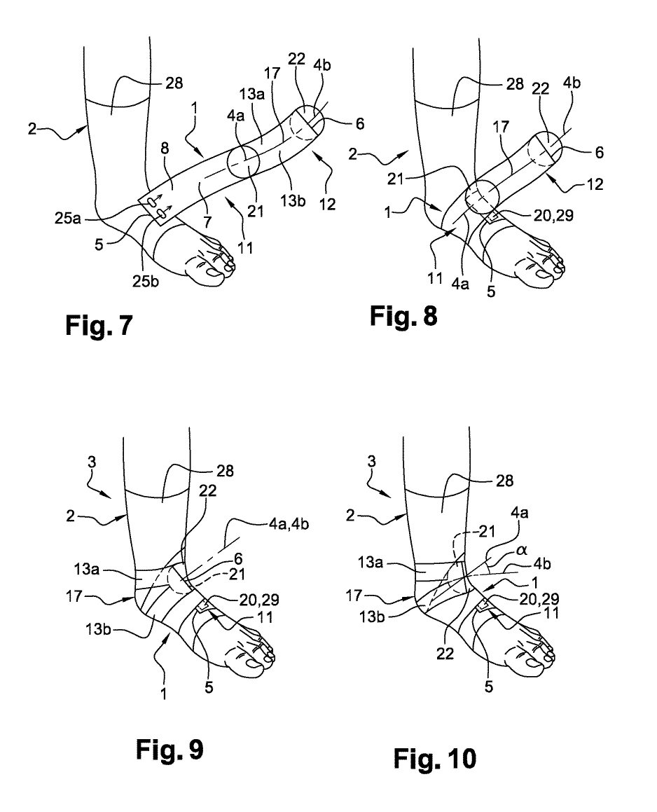

Now, reference is made to FIGS. 7 to 10 which illustrate different steps of setting up of the orthosis 3 on the ankle of a person.

Firstly, the user threads the ankle brace 2, then he assembles the strip 1 on the ankle brace 2 by placing the assembly components 20 arranged on the strip 1 on the complementary assembly component 29 arranged on the ankle brace 2. It may be envisaged that the assembly components 20, 29 are rectangular and substantially with the same dimensions. Thereby, their superposition ensures that the strip 1 is assembled in a correct way on the ankle brace 2 and that the user may begin to wind the strip 1. Furthermore, the fact of having on the strip 1 a rectangular assembly component 20, and a second disc-shaped attachment component 22 allows avoiding that the user assembles the strip 1 upside down on the ankle brace 2, that is to say, by attaching the strip 1 to the ankle brace 2 by the second end 6 and not by the first end 5.

It is fundamental that the strip 1 is correctly positioned around the ankle, otherwise the orthosis 3 would not have the required efficiency. This could even lead to obtaining the effect opposite to that intended, in particular in setting in varus of the foot. In practice, the traction of the strip 1 must be done towards the inside of the foot. But, advantageously, the orthosis 3—mainly both the ankle brace 2 and the strip 1—are indifferently provided for the left foot or for the right foot. It follows that they generally have no clear asymmetry indicating the correct direction of setting up.

Thus, it may be envisaged to provide the strip 1 with fool-proofing means allowing indicating to the user the correct direction of setting up and winding the strip. These fool-proofing means may be located in the vicinity of the first end 5 and on the outer face 8 of the strip 1. They may comprise:

on the strap 13a located above the strap 13b when the slot 17 is on the right, the drawing 25a of a left foot combined to an arrow 26a facing the second end 6 of the strip 1, the heel of this foot 25a being located near the longitudinal edge 14;
and on the strap 13b, the drawing 25b of a right foot combined to an arrow 26b facing the second end 6 of the strip 1, the heel of this foot 25b being located near the longitudinal edge 15.

Once the strip 1 correctly assembled on the ankle brace 2, as illustrated in FIG. 6, the user stretches the strip 1 inwardly, passes it under his foot and returns thereon (FIG. 8). The first attachment component 21 is then located substantially at the instep.

Finally, the user continues to wind the strip 1 by passing it at the back of the leg then at the heel, the latter being housed in the slot 17 which is enlarged due to the spacing of the two longitudinal edges 14, 15 on either side of the heel. It is preferable that the strip 1 is stretched in a substantially constant manner throughout its winding. The strip 1 is then in the position of use, wound on itself and attached to itself by the cooperation between the first and the second attachment components 21, 22 (FIGS. 9 and 10).

Depending on how the user has wound the strip 1, the second portion 12 of the strip 1 may be in different configurations with respect to the first portion 11. Two different configurations are illustrated by way of example:
in FIG. 9, the transverse edge corresponding to the second end 6 of the strip 1 is substantially located in a sagittal plane of the user, for example substantially median with respect to the foot. In other words, the second attachment component 22 is assembled to the first attachment component 21 so that the portion 4b of the longitudinal axis of symmetry 4 of the strip 1 at the second attachment component 22 is substantially parallel to the portion 4a of the longitudinal axis of symmetry 4 of the strip 1 at the first attachment component 21;
in FIG. 10, the second attachment component 22 is assembled to the first attachment component 21 so that the portion 4b of the longitudinal axis of symmetry 4 of the strip 1 at the second attachment component 22 makes an angle α with the portion 4a of the longitudinal axis of symmetry 4 of the strip 1 at the first attachment component 21.

Despite this difference in relative angular positioning, due to the disc shape of the attachment components 21, 22, there is always an almost perfect superposition of the attachment components 21, 22. For this superposition, the user does not need to act on the second portion 12 of the strip 1 for varying the angle α, in particular to reduce it, which would cause the aforementioned problems.

Thus, the invention allows an intuitive positioning of the strip, and ensures that the forces exerted on the strip, hence on the member of the user, are better distributed, by avoiding local tensions in the strip which damages the holding efficiency and the comfort.

It goes without saying that the invention is not limited to the embodiment described hereinabove by way of example but it comprises all the equivalent techniques and variants of the described means as well as their combinations.

1. An elastic strip for holding the ankle of a person, the strip having an inner face and an outer face and comprising:
a first portion extending from a first end of the strip and which is intended to be wound substantially around the foot;
a second portion extending in the extension of the first portion to a second end of the strip and which is intended to be wound substantially around the ankle;
a first attachment component, located on the outer face of the strip, substantially at the junction between the first portion and the second portion;
a second attachment component intended to cooperate with the first attachment component, located on the inner face of the strip, in the vicinity of the second end of the strip;
wherein the first and the second attachment components are substantially disc-shaped, the second attachment component having a diameter lower than or equal to the diameter of the first attachment component.

2. The strip according to claim 1, wherein the second attachment component diameter is greater than 0.6 times the first attachment component diameter.

3. The strip according to claim 1, wherein the first and second attachment components have substantially identical diameters.

4. The strip according to claim 3, wherein the strip is substantially rectangular, and in that the diameter of the first and second attachment components is substantially equal to the width of the strip.

5. The strip according to any of claim 1, wherein the width of the second portion shifts from a value in the vicinity of the first portion to a value lower than in the vicinity of the second end, and in that the diameter of the first attachment component is substantially equal to I, and in that the diameter of the second attachment component is substantially equal to I′.

6. The strip according to any of claim 1, wherein the first and second attachment components are hook and loop type of a Velcro®-type system.

7. The strip according to any of claim 1, wherein the strip comprises, in the vicinity of its first end, and on its inner face, an assembly component on an ankle brace including a tubular element made of elastic material.

8. The strip according to any of claim 1, wherein the second portion of the strip has a longitudinal opening intended to accommodate the heel.

9. The strip according to any of claim 1, wherein the second portion of the strip is substantially flat in the unstressed condition.

10. The strip according to any of claim 1, wherein it is formed by two longitudinal straps disposed side by side so that a longitudinal edge of a strap is adjacent to a longitudinal edge of the other strap at least on the first portion of the strip, said longitudinal edges being assembled to each other over substantially the entire first portion of the strip, the straps being assembled to each other in the vicinity of the free end of the second portion and being free relative to each other over substantially all the remainder of the second portion to form a slot therebetween.

11. The strip according to any of claim 1, wherein the strip comprises, in the vicinity of the first end and on the outer face of the strip, fool-proofing means allowing indicating to the user the correct direction of setting up the strip.

12. An ankle orthosis comprising an ankle brace including a tubular element made of resilient material intended to be threaded over the ankle of a person, wherein the ankle orthosis further comprises a strip according to claim 1, intended to be wound over the ankle brace.

13. The ankle orthosis according to claim 12, wherein the strip is substantially rectangular, and in that the diameter of the first and second attachment components is substantially equal to the width of the strip, and in that the ankle brace comprises a complementary assembly component arranged on a front area intended to be placed on the top of the foot.
